(19) United States
(12) Patent Application Publication
(10) Pub. No.: US 2015/0352030 A1
(43) Pub. Date: Dec. 10, 2015

(54) COMPOSITION

(71) Applicants: UNILEVER PLC, London (GB); UNILEVER N.V., Rotterdam (NL); CONOPCO, INC., D/B/A UNILEVER, Englewood Cliffs, NJ (US)

(72) Inventors: Stephen Norman BATCHELOR, Chester (GB); Adam John LIMER, Northwich (GB); Xuezhi TANG, Shanghai (CN)

(73) Assignee: Conopco, Inc., d/b/a UNILEVER, Englewood Cliffs, NJ (US)

(21) Appl. No.: 14/760,596

(22) PCT Filed: Jan. 3, 2014
(86) PCT No.: PCT/EP2014/050057

§ 371 Date: Jul. 13, 2015

(30) Foreign Application Priority Data
Jan. 15, 2013 (CN) PCT/CN2013/070475

(51) Int. Cl. A61K 8/84; A61Q 5/10; C09B 69/10

(52) U.S. Cl. CPC A61K 8/84; C09B 69/106; A61Q 5/10; A61K 2800/4324

(57) ABSTRACT

A hair colouring composition comprising analkylated polyethyleneimine dye, wherein the alkylated polyethyleneimine dye comprises a polyethyleneimine having covalently bound to the nitrogen groups of the polyethyleneimine: (i) a negatively charged reactive dye and; (ii) a linear carbon chain comprising at least 7 carbon atoms.

The present invention relates to alkylated polyethleneimine dyes in hair care colouring compositions.

U.S. Pat. No. 4,182,612 (Gillette) discloses polyethyleneimine cationic polymers covalently linked to dyes for the colouration of hair. The dyes used do not carry negatively charged substituents.

WO2011/113680 (Unilever) discloses polyethylene and polypropylene imines bound to reactive dyes for the colouration of hair.

However; there remains the need to mitigate skin staining caused by reactive dye hair colouring polymers.

### SUMMARY OF THE INVENTION

The present invention relates to a hair colouring composition comprising an alkylated polyethyleneimine dye, wherein the alkylated polyethyleneimine dye consisting essentially of a polyethyleneimine having covalently bound to the nitrogen groups of the polyethyleneimine:

(i) a negatively charged reactive dye and;
(ii) a linear carbon chain comprising at least 7 carbon atoms.

In a second aspect the invention relates to a method of colouring hair comprising the step of applying the above composition to the hair.

In a third aspect the invention provides the use a polyethyleneimine described above for colouring hair.

In a fourth aspect the invention relates to an alkylated polyethyleneimine dye comprising a polyethyleneimine having covalently bound to the nitrogen groups of the polyethyleneimine:

(i) a negatively charged reactive dye and;
(ii) a linear carbon chain comprising at least 7 carbon atoms.

### DETAILED DESCRIPTION OF THE INVENTION

Unless specified otherwise, all wt % values quoted hereinafter are percentages by weight based on total weight of the hair treatment composition.

Polyethyleneimine has been abbreviated in the text to PEI.

### Dye Polymer

The present invention relates to a polyethyleneimine having covalently bound to the nitrogen groups of the polyethyleneimine:

(i) a negatively charged reactive dye and;
(ii) linear carbon chain comprising at least 7 carbon atoms.

In the context of the present invention the dye carries a negatively charged group before and after addition to the polyethyleneimine.

Preferably the PEI before reaction with the alkyl moieties contains at least 4 primary or secondary amines.

Preferably PEI before reaction has from 6 to 100 nitrogen atoms, more preferably from 12 to 40 nitrogen atoms, most preferably from 18 to 36.

Specific examples of polyethylene imines are PEI-300, PEI-500, PEI-600, PEI-700, PEI-800, PEI-1000, PEI-1500, PEI-1800, PEI-2000, PEI-2500, PEI-5000, PEI-10000, PEI-25000, PEI 50000 and PEI-70000, wherein the integer represents the number average molecular weight (M_{w}) of the polymer. PEI's which are designated as such are available through Aldrich.

Preferably the PEI is not alkoxylated.

Alkylation is the covalent bonding of an alkyl group to the polyethyleneimine. The alkyl group is a linear carbon chain with at least 7 carbon atoms, preferably C₈ to C₂₆ carbon atoms, more preferably C₁₂ to C₂₂ carbon atoms, most preferably C₁₂, C₁₄ and C₁₆ carbon chain. In a highly preferred embodiment the carbon chain is an unsaturated alkyl group. The alkyl group does not have an aryl substituent but may carry non-aromatic and non-alkyl substituents, for example OH, CN. The alkyl group does not bare an amine functionality. In a further embodiment the alkyl group may include a carbonyl functionality when this is directly connected to an amine of the PEI. Preferably the alkyl group is not substituted by more than one non-alkyl substituents.

The alkylation is preferably carried out by treating the amine(s) of the PEI with an electrophilic alkylation agent.

PEI may be alkylated using a wide variety of alkylating agents. Suitable alkylating agents include dialkyl sulfates, alcohols, alkyl halides, epoxides, olefins, and carbonyl compounds. These reactions are described in Ullmann's Encyclopedia of Industrial Chemistry, Acylation and Alkylation, Michael Röper, Eugen Gehrer, Thomas Narbeshuber & Wolfgang Siegel (John Wiley ISBN: 9783527306732) and in KIRK-OTHMER ENCYCLOPEDIA OF CHEMICAL TECHNOLOGY, Vol 8, DIAMINES AND HIGHER AMINES, ALIPHATIC (John Wiley ISBN: 9780471238966).

Preferred common alkylating agents include dialkyl sulphates, alkyl bromides and iodides, alkyl tosylates, alkyl epoxides, and fatty acids.

The covalent coupling Fatty acids to the polymer is preferably activated with a carbodiimide. Preferred carbodiimides are dicyclohexylcarbodiimide and N,N′-Diisopropylcarbodiimide.

Simplified reaction scheme for the epoxide and fatty acid coupling are

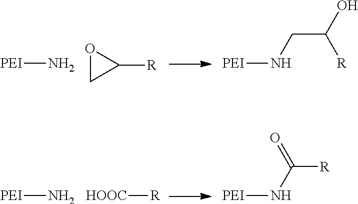

R represents an alkyl chain. For the epoxide the length of the alkyl chain is the length of R plus 2, to account for the —CH₂—CH(OH)— group.

For the fatty acid the length of the alkyl chain includes the carbonyl group. For example the binding of CH₃(CH₂)₁₂COOH to PEI, provides a chain of 14.

Fatty acids are preferably saturated

Preferably the alkyl group is directly bound to the PEI or bound via an acid amide group.

In the context of the present invention the term covalently bound with respect to the alkyl group means that the alkyl group is directly attached to the PEI.

Thus In the context of the present invention the term alkyl group does not include ethers.

Preferably the PEI is branched and has M_{w} of 500 to 1800, most preferably 700 to 1600.

Preferably there are least one mol equivalents of alkyl groups per one mole of PEI.

Preferably there are one to 10 mol equivalents of alkyl groups per one mole of PEI, more preferably 1 to 5, most preferably 1 to 2.

An example of an alkylated PEI structure is

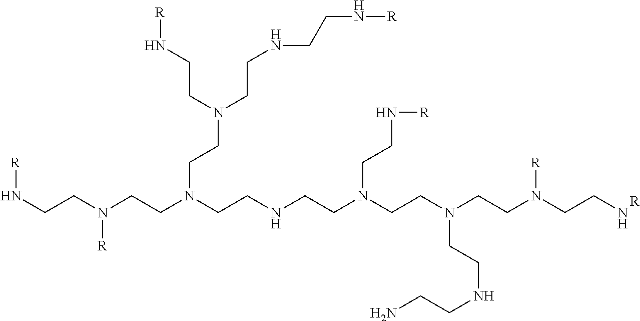

Where R represents an alkyl group. The alkylated PEI has 7 alkyl groups in the above example. The original PEI had 11, primary and secondary Nitrogens. The alkylated PEI has 7/11*100=63.6 mol % of the primary and secondary nitrogens of the original PEI are alkylated.

Suitable reactive dyes for use with PEI are described in Industrial Dyes (K. Hunger ed, Wiley VCH 2003). Many Reactive dyes are listed in the colour index (Society of Dyers and Colourists and American Association of Textile Chemists and Colorists).

Reactive groups are preferably selected from heterocyclic reactive groups and, a sulfooxyethylsulfonyl reactive group (—SO₂CH₂CH₂OSO₃Na), which is converts to a vinylsulfone in alkali. The heterocyclic reactive groups are preferably nitrogen contains aromatic rings bound to a halogen or an ammonium group or a quaternary ammonium group which react with NH₂ or NH groups of the polymers to form a covalent bonds. The halogen is preferred, most preferably Cl or F.

Preferably, the reactive dye comprises a reactive group selected from dichlorotriazinyl, difluorochloropyrimidine, monofluorotrazinyl, dichloroquinoxaline, vinylsulfone, difluorotriazine, monochlorotriazinyl, bromoacrlyamide and trichloropyrimidine.

Preferably the dye is sulphonated.

The reactive group may be linked to the dye chromophore via an alkyl spacer for example: dye-NH—CH₂CH₂-reactive group.

Especially preferred heterocylic reactive groups are:

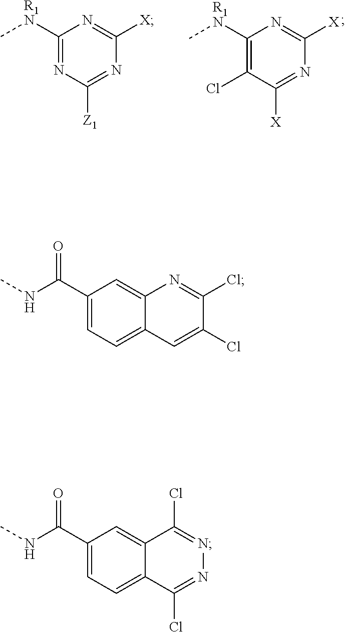

Wherein R₁ is selected from H or alkyl, preferably H.

X is selected from F or Cl

When X═Cl, Z₁ is selected from —Cl, —NR₂R₃, —OR₂, —SO₃Na

When X═F, Z₁ is selected from —NR₂R₃

R₂ and R₃ are independently selected from H, alkyl and aryl groups. Aryl groups are preferably phenyl and are preferably substituted by —SO₃Na or —SO₂CH₂CH₂OSO₃Na. Alkyl groups are preferably selected from methy and ethyl.

The phenyl groups may be further substituted with suitable uncharged organic groups, preferably with a molecular weight lower than 200. Preferred groups include —CH₃, —C₂H₅, and —OCH₃.

The alkyl groups may be further substituted with suitable uncharged organic groups, preferably with a molecular weight lower than 200. Preferred groups include —CH₃, —C₂H₅, —OH, —OCH₃, —OC₂H₄OH.

Most preferred heterocylic reactive groups are selected from

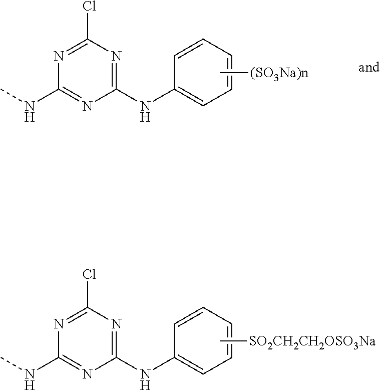

Where n=1 or 2, preferably 1.

Preferably the reactive dye contains more than one reactive group, preferably two or three.

Preferably, the reactive dye comprises a chromophore selected from azo, anthraquinone, phthalocyanine, formazan and triphendioaxazine.

Where the dye is an azo dye it is preferred that the azo dye is not an azo-metal complex dye.

Preferably the reaction of the polyamine and the reactive dye to form the dye polymers, takes place in water at alkaline pH, preferable pH=10 to 11.5, at temperature of 40-100° C. for 1 to 3 hours after the dye is added to the solution. Thereafter the solution is cooled to room temperature and neutralised to pH=7 within 1 to 2 hours. The level of polyamine in the reaction solution is preferable from 2 to 50 wt %, more preferably from 5 to 20 wt %. These conditions minimise the production of hydrolysed dye.

Examples of reactive dyes include reactive black 5, reactive blue 19, reactive red 2, reactive blue 171, reactive blue 269, reactive blue 11, reactive yellow 17, reactive, reactive orange 4, reactive orange 16, reactive green 19, reactive brown 2, reactive brown 50.

Reactive blue dyes are preferably selected from anthraquinone, mono azo, bis-azo, triphenodioxazine, and phthalocyanine, more preferably anthraquinone, bis-azo, and triphenodioxazine, most preferably bis-azo and triphenodioxazine.

A preferred blue bis-azo dye is of the form

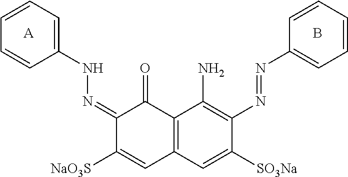

Where one or both of the A and B rings are substituted by a reactive group.

The A and B rings may be further substituted by sulphonate groups (SO₃Na).

The A and B rings may be further substituted with suitable uncharged organic groups, preferably with a molecular weight lower than 200. Preferred uncharged organic groups are —CH₃, —C₂H₅, and —OCH₃.

Preferred blue bis-azo dye dyes are Reactive Black 5, Reactive Blue 171, Reactive Blue 154, Reactive Blue 184, Reactive Blue 207, Reactive Blue 214, Reactive Blue 217, Reactive Blue 203, Reactive Blue 225, Levafix Navy CA, Procion Navy H-EXL, Reactive Blue 176, Reactive Blue 109, Reactive Blue 230, Reactive Blue 225, Reactive Blue 222, Reactive Blue 250 and Reactive Blue 281.

A preferred blue anthraquinone dye is of the form:

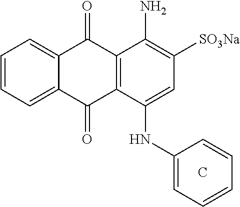

Where the C ring is substituted by a reactive group. The dye may be further substituted with sulphonate groups (SO₃Na) and suitable uncharged organic groups, preferably with a molecular weight lower than 200. Preferred uncharged organic groups are —CH₃, —C₂H₅, and —OCH₃.

A preferred blue triphenodioxazine dye is of the form:

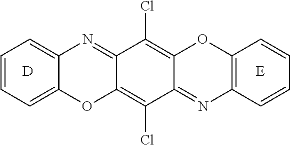

Where the D and E rings are substituted by a reactive groups. Preferably the D and E rings are further substituted by sulphonate groups (SO₃Na).

Examples of reactive blue dyes are reactive blue 2, reactive blue 4, reactive blue 5, reactive blue 7, reactive blue 15, reactive blue 19, reactive blue 27, reactive blue 29, reactive blue 49, reactive blue 50, reactive blue 74, reactive blue 94, reactive blue 246, reactive blue 247, reactive blue 247, reactive blue 166, reactive blue 109, reactive blue 187, reactive blue 213, reactive blue 225, reactive blue 238, reactive blue 256. Further structures are exemplified below:

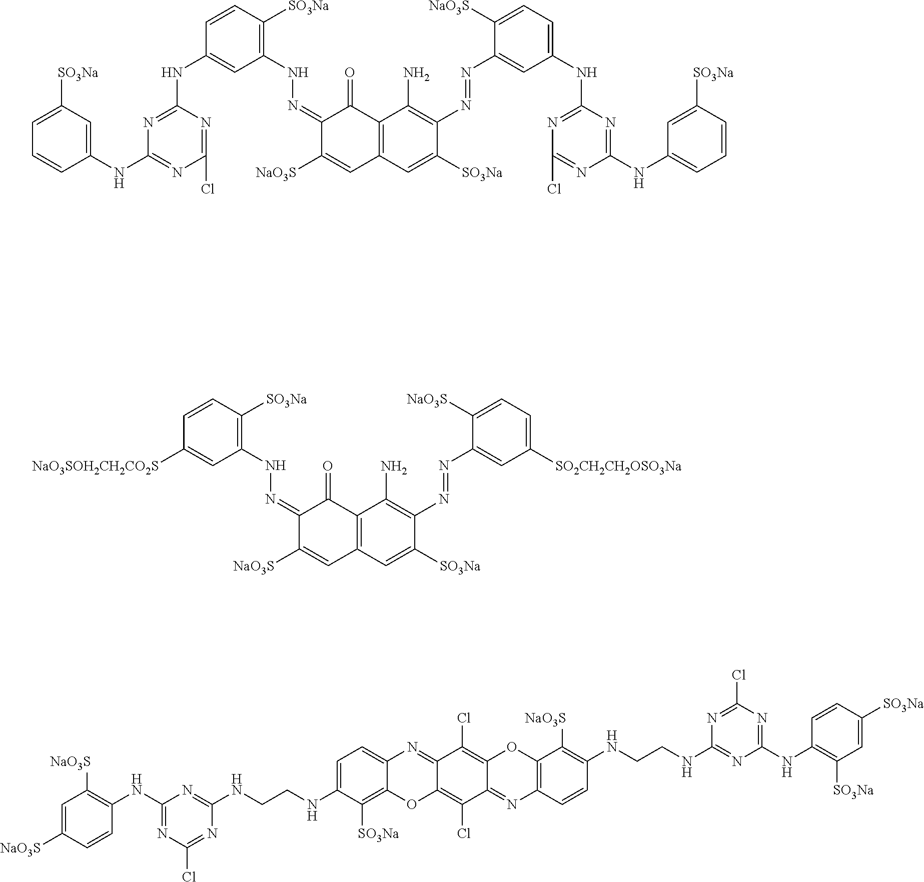

Reactive Red dyes are preferably selected from mono-azo and bis-azo dyes.

A preferred reactive red azo dye is of the form

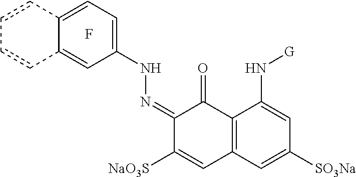

Where the F ring is optionally extended to form a naphthyl group and is optionally substituted by groups selected from sulphonate groups (SO₃Na) and a reactive group.

G is selected from a reactive group, H, or alky group. A reactive group must be present on the dye.

Examples of reactive red dyes are reactive red 2, reactive red 3, reactive red 4, reactive red 8, reactive red 9, reactive red 12, reactive red 13, reactive red 17, reactive red 22, reactive red 24, reactive red 29, reactive red 33 reactive red 139, reactive red 198 and reactive red 141.

Reactive yellow and orange dyes are preferably selected from mono-azo dyes. Examples of reactive yellow and orange dyes are reactive yellow 1, reactive yellow 2, reactive yellow 3, reactive yellow 16, reactive yellow 17, reactive yellow 25, reactive yellow 39, reactive orange 107, reactive yellow 176 and reactive yellow 135.

Combination of reactive dyes may be used to obtain a wide colour palette with use of a limited number of dyes. Preferably, a trichromate system consisting of a mixture of three reactive dyes. Preferably, the trichromate system contains a combination of a reactive blue or a reactive black dye, a reactive red and a reactive yellow dye. For example reactive black 5, reactive yellow 176 and reactive red 239; reactive blue 171, reactive yellow 176 and reactive red 141.

Preferably the mole ratio of PEI to dye is from 1:1 to 2:1.

Preferably, the dye polymer is obtainable by reacting the polymer with from 0.1 to 40 wt % reactive dye, most preferably from 5 to 20 wt %.

An example structure of an alkylated polyethyleneimine dye is:

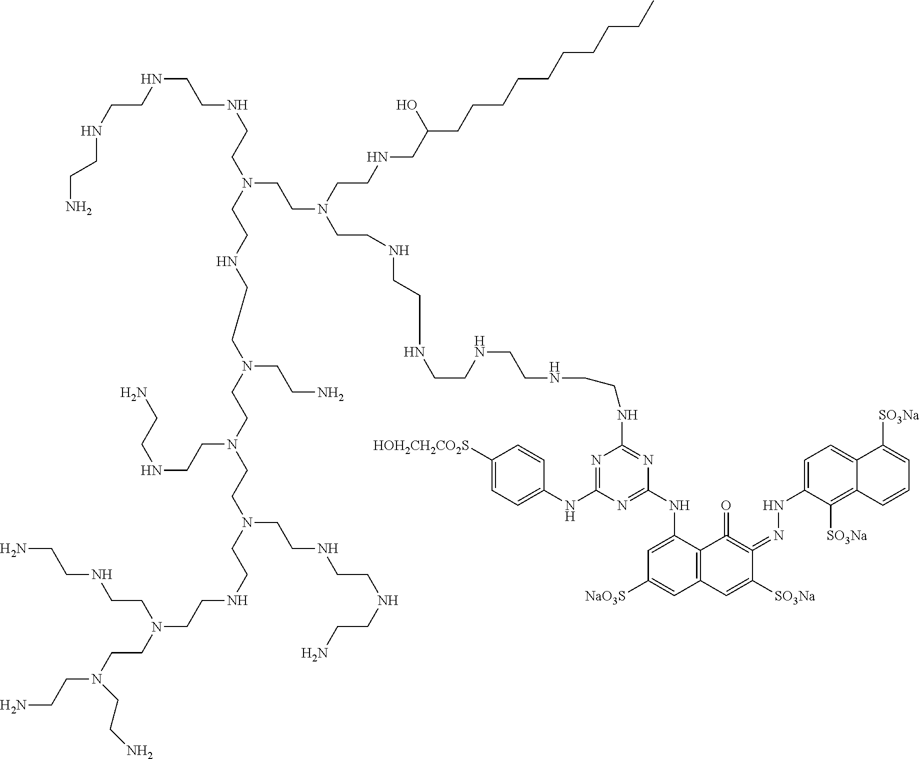

### Composition

It is preferable that the hair colourants of the present application are formulated as conditioning compositions.

Preferably, the water used to formulate all compositions has a French hardness of from 0 to 36 degrees, more preferably 0 to 24 degrees, most preferably from 0 to 2 degrees.

Preferably, the water used to formulate all compositions contains less than 1 ppm of chlorine based bleaching agents such as chlorine dioxide or hypochlorite. Most preferably less than 50 ppb.

### Silicone Conditioning Agents

The compositions of the invention can contain, emulsified droplets of a silicone conditioning agent, for enhancing conditioning performance.

Suitable silicones include polydiorganosiloxanes, in particular polydimethylsiloxanes which have the CTFA designation dimethicone. Also suitable for use compositions of the invention (particularly shampoos and conditioners) are polydimethyl siloxanes having hydroxyl end groups, which have the CTFA designation dimethiconol. Also suitable for use in compositions of the invention are silicone gums having a slight degree of cross-linking, as are described for example in WO 96/31188.

The viscosity of the emulsified silicone itself (not the emulsion or the final hair conditioning composition) is typically at least 10,000 cst at 25° C. the viscosity of the silicone itself is preferably at least 60,000 cst, most preferably at least 500,000 cst, ideally at least 1,000,000 cst. Preferably the viscosity does not exceed 10⁹ cst for ease of formulation.

Emulsified silicones for use in the shampoo compositions of the invention will typically have an average silicone droplet size in the composition of less than 30, preferably less than 20, more preferably less than 10 μm, ideally from 0.01 to 1 μm. Silicone emulsions having an average silicone droplet size of 0.15 μm are generally termed microemulsions.

Emulsified silicones for use in the conditioner compositions of the invention will typically have an size in the composition of less than 30, preferably less than 20, more preferably less than 15. Preferably, the average silicone droplet is greater than 0.5 μm, more preferably greater than 1 μm, ideally from 2 to 8 μm.

Silicone particle size may be measured by means of a laser light scattering technique, for example using a 2600D Particle Sizer from Malvern Instruments.

Examples of suitable pre-formed emulsions include Xiameter MEM 1785 and microemulsion DC2-1865 available from Dow Corning. These are emulsions/microemulsions of dimethiconol. Cross-linked silicone gums are also available in a pre-emulsified form, which is advantageous for ease of formulation

A further preferred class of silicones for inclusion in shampoos and conditioners of the invention are amino functional silicones. By “amino functional silicone” is meant a silicone containing at least one primary, secondary or tertiary amine group, or a quaternary ammonium group. Examples of suitable amino functional silicones include: polysiloxanes having the CTFA designation “amodimethicone”. Specific examples of amino functional silicones suitable for use in the invention are the aminosilicone oils DC2-8220, DC2-8166 and DC2-8566 (all ex Dow Corning).

Suitable quaternary silicone polymers are described in EP-A-0 530 974. A preferred quaternary silicone polymer is K3474, ex Goldschmidt.

Also suitable are emulsions of amino functional silicone oils with non ionic and/or cationic surfactant. Pre-formed emulsions of amino functional silicone are also available from suppliers of silicone oils such as Dow Corning and General Electric. Specific examples include DC939 Cationic Emulsion and the non-ionic emulsions DC2-7224, DC2-8467, DC2-8177 and DC2-8154 (all ex Dow Corning).

With some shampoos it is preferred to use a combination of amino and non amino functional silicones

The total amount of silicone is preferably from 0.01 wt % to 10% wt of the total composition more preferably from 0.1 wt % to 5 wt %, most preferably 0.5 wt % to 3 wt % is a suitable level.

### Non-Silicone Oily Conditioning Components

Compositions according to the present invention may also comprise a dispersed, non-volatile, water-insoluble oily conditioning agent. By “insoluble” is meant that the material is not soluble in water (distilled or equivalent) at a concentration of 0.1% (w/w), at 25° C.

Suitable oily or fatty materials are selected from hydrocarbon oils, fatty esters and mixtures thereof. Straight chain hydrocarbon oils will preferably contain from about 12 to about 30 carbon atoms. Also suitable are polymeric hydrocarbons of alkenyl monomers, such as C₂-C₆ alkenyl monomers.

Specific examples of suitable hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, and mixtures thereof. Branched-chain isomers of these compounds, as well as of higher chain length hydrocarbons, can also be used.

Suitable fatty esters are characterised by having at least 10 carbon atoms, and include esters with hydrocarbyl chains derived from fatty acids or alcohols, Monocarboxylic acid esters include esters of alcohols and/or acids of the formula R′COOR in which R′ and R independently denote alkyl or alkenyl radicals and the sum of carbon atoms in R′ and R is at least 10, preferably at least 20. Di- and trialkyl and alkenyl esters of carboxylic acids can also be used.

Particularly preferred fatty esters are mono-, di- and triglycerides, more specifically the mono-, di-, and tri-esters of glycerol and long chain carboxylic acids such as C₁-C₂₂ carboxylic acids. Preferred materials include cocoa butter, palm stearin, sunflower oil, soyabean oil and coconut oil.

The oily or fatty material is suitably present at a level of from 0.05 wt % to 10 wt %, preferably from 0.2 wt % to 5 wt %, more preferably from about 0.5 wt % to 3 wt %.

### Cationic Conditioning Surfactants

Preferably the composition comprises a cationic surfactant.

Suitable conditioner compositions will typically comprise one or more conditioning surfactants which are cosmetically acceptable and suitable for topical application to the hair.

Suitable conditioning surfactants include those selected from cationic surfactants, used singly or in admixture. Preferably, the cationic surfactants have the formula N⁺R¹R²R³R⁴ wherein R¹, R², R³ and R⁴ are independently (C₁ to C₃₀) alkyl or benzyl. Preferably, one, two or three of R¹, R², R³ and R⁴ are independently (C₄ to C₃₀) alkyl and the other R¹, R², R³ and R⁴ group or groups are (C₁-C₆) alkyl or benzyl. More preferably, one or two of R¹, R², R³ and R⁴ are independently (C₆ to C₃₀) alkyl and the other R¹, R², R³ and R⁴ groups are (C₁-C₆) alkyl or benzyl groups. Optionally, the alkyl groups may comprise one or more ester (—OCO— or —COO—) and/or ether (—O—) linkages within the alkyl chain. Alkyl groups may optionally be substituted with one or more hydroxyl groups. Alkyl groups may be straight chain or branched and, for alkyl groups having 3 or more carbon atoms, cyclic. The alkyl groups may be saturated or may contain one or more carbon-carbon double bonds (eg, oleyl). Alkyl groups are optionally ethoxylated on the alkyl chain with one or more ethyleneoxy groups.

Suitable cationic surfactants for use in conditioner compositions according to the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (eg, Arquad 2HT/75 from Akzo Nobel), cocotrimethylammonium chloride, PEG-2-oleammonium chloride and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in conditioners according to the invention is cetyltrimethylammonium chloride, available commercially, for example as GENAMIN CTAC, ex Hoechst Celanese. Another particularly useful cationic surfactant for use in conditioners according to the invention is behenyltrimethylammonium chloride, available commercially, for example as GENAMIN KDMP, ex Clariant.

Another example of a class of suitable cationic surfactants for use in the invention, either alone or together with one or more other cationic surfactants, is a combination of (i) and (ii) below:

(i) an amidoamine corresponding to the general formula (I):

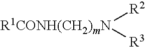

in which R¹ is a hydrocarbyl chain having 10 or more carbon atoms, R² and R³ are independently selected from hydrocarbyl chains of from 1 to 10 carbon atoms, and m is an integer from 1 to about 10; and
   (ii) an acid.

As used herein, the term hydrocarbyl chain means an alkyl or alkenyl chain.

Preferred amidoamine compounds are those corresponding to formula (I) in which

R¹ is a hydrocarbyl residue having from about 11 to about 24 carbon atoms, R² and R³ are each independently hydrocarbyl residues, preferably alkyl groups, having from 1 to about 4 carbon atoms, and m is an integer from 1 to about 4.

Preferably, R² and R³ are methyl or ethyl groups.

Preferably, m is 2 or 3, i.e. an ethylene or propylene group.

Preferred amidoamines useful herein include stearamido-propyldimethylamine, stearamidopropyldiethylamine, stearamidoethyldiethylamine, stearamidoethyldimethylamine, palmitamidopropyldimethylamine, palmitamidopropyl-diethylamine, palmitamidoethyldiethylamine, palmitamidoethyldimethylamine, behenamidopropyldimethyl-amine, behenamidopropyldiethylmine, behenamidoethyldiethyl-amine, behenamidoethyldimethylamine, arachidamidopropyl-dimethylamine, arachidamidopropyldiethylamine, arachid-amidoethyldiethylamine, arachidamidoethyldimethylamine, and mixtures thereof.

Particularly preferred amidoamines useful herein are stearamidopropyldimethylamine, stearamidoethyldiethylamine, and mixtures thereof.

Commercially available amidoamines useful herein include:

stearamidopropyldimethylamine with tradenames LEXAMINE S-13 available from Inolex (Philadelphia Pa., USA) and AMIDOAMINE MSP available from Nikko (Tokyo, Japan), stearamidoethyldiethylamine with a tradename AMIDOAMINE S available from Nikko, behenamidopropyldimethylamine with a tradename INCROMINE BB available from Croda (North Humberside, England), and various amidoamines with tradenames SCHERCODINE series available from Scher (Clifton N.J., USA).

A protonating acid may be present. Acid may be any organic or mineral acid which is capable of protonating the amidoamine in the conditioner composition. Suitable acids useful herein include hydrochloric acid, acetic acid, tartaric acid, fumaric acid, lactic acid, malic acid, succinic acid, and mixtures thereof. Preferably, the acid is selected from the group consisting of acetic acid, tartaric acid, hydrochloric acid, fumaric acid, lactic acid and mixtures thereof.

The primary role of the acid is to protonate the amidoamine in the hair treatment composition thus forming a tertiary amine salt (TAS) in situ in the hair treatment composition. The TAS in effect is a non-permanent quaternary ammonium or pseudo-quaternary ammonium cationic surfactant.

Suitably, the acid is included in a sufficient amount to protonate more than 95 mole % (293 K) of the amidoamine present.

In conditioners of the invention, the level of cationic surfactant will generally range from 0.01% to 10%, more preferably 0.05% to 7.5%, most preferably 0.1% to 5% by weight of the total composition.

Conditioners of the invention will typically also incorporate a fatty alcohol. The combined use of fatty alcohols and cationic surfactants in conditioning compositions is believed to be especially advantageous, because this leads to the formation of a lamellar phase, in which the cationic surfactant is dispersed.

Representative fatty alcohols comprise from 8 to 22 carbon atoms, more preferably 16 to 22. Fatty alcohols are typically compounds containing straight chain alkyl groups. Examples of suitable fatty alcohols include cetyl alcohol, stearyl alcohol and mixtures thereof. The use of these materials is also advantageous in that they contribute to the overall conditioning properties of compositions of the invention.

The level of fatty alcohol in conditioners of the invention will generally range from 0.01% to 10%, preferably from 0.1% to 8%, more preferably from 0.2% to 7%, most preferably from 0.3% to 6% by weight of the composition. The weight ratio of cationic surfactant to fatty alcohol is suitably from 1:1 to 1:10, preferably from 1:1.5 to 1:8, optimally from 1:2 to 1:5. If the weight ratio of cationic surfactant to fatty alcohol is too high, this can lead to eye irritancy from the composition. If it is too low, it can make the hair feel squeaky for some consumers.

### Adjuncts

The compositions of the present invention may also contain adjuncts suitable for hair care. Generally such ingredients are included individually at a level of up to 2 wt %, preferably up to 1 wt % of the total composition.

Suitable hair care adjuncts, include perfumes; amino acids, sugars and ceramides and viscosity modifiers.

### Product Form

Compositions of the present invention are formulated into hair colouring compositions which may take a variety of forms, including, for example, mousses, gels, lotions, creams, sprays and tonics. These product forms are well known in the art.

The preferred product is a lotion, cream, mousse or gel.

Preferably, the composition is a rinse off hair treatment composition. A rinse off composition is applied hair preferably to wet hair, and left on the hair for up to I hour, preferably left on the hair for up to 30 minutes before it is removed by rinsing.

Example of the invention will be illustrated with the following non-limiting Examples.

Examples of the invention are illustrated by a number, comparative Examples by a letter.

### EXAMPLES

### Example 1

Polymer A (control): 40 g of a branched polyethylene imine (pH-11) with a M_{w} of 2000 was mixed with 10 g of reactive black 5 in 260 ml of water and stirred at room temperature for 18 hours, then pH adjusted to 7 with HCl. The crude product was dialysed against deionized water.

Polymers 1, 2 and 3: 40 g of a branched polyethylene imine (pH-11) with a M_{w} of 2000 was mixed 4 g of alkyl epoxide, as detailed in table 1, and stirred for 18 hours at room temperature. 10 g of reactive black 5 was then added and stirred at 55° C. for 2 hours, then pH adjusted to 7 with HCl. The crude product was dialysed against deionized water.

TABLE 1

| Polymer | A | B | 1 | 2 |
|---|---|---|---|---|
| PEI 2000 | 40 g | 40 g | 40 g | 40 g |
| 1,2 Epoxy hexane | | 4 g | | |
| 1,2 Epoxy octane | | | 4 g | |
| 1,2 Epoxy hexadecane | | | | 4 g |
| Reactive Black 5 | 10 g | 10 g | 10 g | 10 g |

A bleached Chinese white human hair swatch weighing 0.7 g was placed in an aqueous solution at pH=9, containing 0.15 wt % of dye-polymer. The solution was agitated for 5 minutes at room temperature and the hair swatch removed, rinsed under running water and dried. The colour of the swatch was then measured using a reflectometer and expressed as the CIE L*a*b* values. The dye-polymers gave a blue colour to the hair, and this was measured as the decrease in L* value relative to an un-dyed swatch:

ΔL=L(dyed)−L(undyed)

The relative substantivity of the polymers was calculated as Δb/Δb(polymer A) and the results shown below:

TABLE 2

| | substantivity |
|---|---|
| Polymer A (reference) | 1 |
| Polymer B (C6) (reference) | 1.1 |
| Polymer 1 (C8) | 0.9 |
| Polymer 2 (C16) | 1.0 |

All polymers dye the hair.

### Example 2

Polymers were assessed for their skin staining potential from a conditioner base formulation. A hair conditioner of the formulation below was produced by a standard process:

TABLE 3

| Ingredient | Wt % |
|---|---|
| Water | To 100 |
| Lactic acid | 0.38 |
| Methyl parahydroxybenzoate | 0.2 |
| Stearyl alcohol | 5 |
| Behenyltrimmonium chloride | 0.87 |
| LexamineS-13 (100% TAS) | 1.25 |
| Silicone emulsion | 2.5 |
| Perfume | 0.5 |
| Dye-polymer | 2 |

The following protocol was used:
1. Prepare 3 strips of pig skin 6.0 cm×2.0 cm per treatment
2. Measure baseline Lab levels and photograph
3. Apply 0.15 g of shampoo and gently massage onto skin for 30 secs (as would be case in vivo)
4. Incubate at RT for 90 sec
5. Rinse for 30 seconds under running tap water (40° C.)
6. Dab with tissue to remove excess water
7. Apply 0.15 g of conditioner and gently massage onto skin for 60 secs (as would be case in vivo)
8. Incubate at RT for 180 sec
9. Rinse for 60 seconds under running tap water (40° C.)
10. Dab with tissue to remove excess water.
11. Measure Lab levels & photograph.
12. Repeat 3-6, and measure Lab levels & photograph.

The colour of the pig skin was then measured using a reflectometer and expressed as the CIE L*a*b* values. The dye-polymers gave a dark blue shade to the skin when excessive deposition had occurred. This was measured as the change in L* value relative to an undyed pig skin:

ΔL=L(dyed)−L(undyed)

A polymeric dye with low skin staining will have the smallest change in L* verses the untreated pig skin.

TABLE 4

| | ΔL |
|---|---|
| Polymer A (reference) | 34.6 |
| Polymer B (C6) (reference) | 35.8 |
| Polymer 1 (C8) | 16.5 |
| Polymer 2 (C16) | 8.0 |

The results above demonstrate that the longer chain C8 and C16 linear alkylated polymers reduced skin staining.

1. A hair colouring composition comprising an alkylated polyethyleneimine dye, wherein the alkylated polyethyleneimine dye comprises a polyethyleneimine having covalently bound to the nitrogen groups of the polyethyleneimine:
(i) a negatively charged reactive dye and;
(ii) a linear carbon chain comprising at least 7 carbon atoms.

2. A hair colouring composition according to claim 1 in which the linear carbon chain has a carbon chain length from C₁₂ to C₂₂.

3. A hair colouring composition according to claim 2 in which the linear carbon chain has a carbon chain length from C₁₂ to C₁₆.

4. A hair colouring composition according to claim 1 in which the linear carbon chain is saturated.

5. A hair colouring composition according to claim 1 in which the polyethyleneimine of the alkylated polyethyleneimine dye has from 6 to 100 nitrogen atoms.

6. A hair colouring composition according to claim 1 in which the polyethyleneimine of the alkylated polyethyleneimine dye has from 12 to 40 nitrogen atoms.

7. A hair colouring composition according to claim 1 in which the reactive dye of the alkylated polyethyleneimine dye comprises a reactive group selected from dichlorotriazinyl, difluorochloropyrimidine, monofluorotrazinyl, dichloroquinoxaline, vinylsulfone, difluorotriazine, monochlorotriazinyl, bromoacrlyamide and trichloropyrimidine.

8. A hair colouring composition according to claim 1 in which the reactive dye of the alkylated polyethyleneimine dye comprises a chromophore selected from azo, anthraquinone, phthalocyanine, formazan and triphendioxazine.

9. A hair colouring composition according to claim 1 in which the reactive dye of the alkylated polyethyleneimine dye is sulphonated.

10. A hair colouring composition according to claim 1 in which mole ratio of PEI to dye is from 1:1 to 2:1.

11. A hair colouring composition according to claim 1 in which the level of alkylated polyethyleneimine dye is from 0.01 to 5 wt % of the total composition.

12. Method for dying hair comprising the steps of applying to the hair a composition as described in claim 1.

13. Use of the alkylated polyethylenimine dye described in claim 1 for colouring hair.

14. An alkylated polyethyleneimine dye comprising a polyethyleneimine having covalently bound to the nitrogen groups of the polyethyleneimine:
(i) a negatively charged reactive dye and;
(ii) a linear carbon chain comprising at least 7 carbon atoms.
